(19) United States
(12) Patent Application Publication
(10) Pub. No.: US 2004/0082593 A1
(43) Pub. Date: Apr. 29, 2004

(54) Infusion of ciprofloxacin having reduced acid content and being stable in storage

(76) Inventors: Klaus Sommermeyer, Rosbach (DE); Hans-Jorg Muller, Braunfels (DE); Tilo Hniopek, Friedberg (DE); Bernd Eschenbach, Hattersheim (DE)

Correspondence Address: James P Zellers, Marshall Gerstein & Borun, 6300 Sears Tower, 233 South Wacker Drive, Chicago, IL, 60606-6357, US

(21) Appl. No.: 10/381,839

(22) PCT Filed: Sep. 27, 2001
(86) PCT No.: PCT/EP01/11189

(30) Foreign Application Priority Data
Sep. 29, 2000 (DE) 100 48 510.3

(51) Int. Cl. A61K 31/496

(52) U.S. Cl. 514/253.08

(57) ABSTRACT

The invention relates to aqueous infusion solutions of 1-cyclopropyl-6-fluoro-1,4-dihydro-4-oxo-7-(1-piperazinyl)-quinoline-3-carboxylic acid (ciprofloxacin) which are stable in storage and which can be obtained by mixing between 0.015 and 0.5 g of active ingredient for 100 ml of aqueous solution with sulphuric acid or sodium hydrogen sulfate in a quantity equal to or less than 0.96 mole per mole of active ingredient, sufficient for dissolving the active ingredient and stabilizing the solution. The invention also relates to methods for producing the infusions and the use thereof. The inventive infusions enable the acid content to be reduced, are more stable in storage with the same acid content than other known solutions, and enable a larger proportion of secondary components to be tolerated in the active ingredient than previous standard infusions of ciprofloxacins.

The present invention relates to infusions of 1-cyclo-propyl-6-fluoro-1,4-dihydro-4-oxo-7-(1-piperazinyl)-quinoline-3-carboxylic acid which are stable on storage and are obtainable by mixing 0.015 to 0.5 g of said active ingredient per 100 ml of aqueous solution with a physiologically tolerated compound in an amount sufficient to dissolve the active ingredient and to stabilize the solution. The invention further relates to the process for the production and to the use of such infusions.

In particular, the invention describes both infusions ready for use and other dosage forms which are conveyed into such infusions before administration, where the active ingredient 1-cyclopropyl-6-fluoro-1,4-dihydro-4-oxo-7-(1-piperazinyl)quinoline-3-carboxylic acid is known as ciprofloxacin.

EP-A-0 049 355 protects inter alia medicaments with a content of 7-amino-1-cyclopropyl-4-oxo-1,4-dihydro-naphtyridine-3-carboxylic acid. EP-A-0 078 362 protects 1-cyclopropyl-6-fluoro-1,4-dihydro-4-oxo-7-piperazinoquinoline-3-carboxylic acids. The active ingredients disclosed in the two EPs have high antibacterial effects and are suitable as medicaments for controlling bacterial infections in humans and animals.

However, the known compounds are unsuited or very little suited for producing infusions and/or injections because, for example, the pH and/or the solubility and/or the stability, especially in relation to sedimentations, of the infusions and/or injections ready for use do not comply with the pharmaceutical requirements to be met by such solutions.

DE 33 33 719 A1 discloses solutions of lactic acid salts of piperazinylquinolone- and/or -azaquinolonecarboxylic acids which, besides said lactic acid salts and, if appropriate, customary exipients, additionally contain at least one acid which does not lead to precipitates. The acids which do not lead to precipitates include, according to DE 33 33 719 A1, lactic acid, methanesulfonic acid, propionic acid or succinic acid, although lactic acid is preferred by far. The lactic acid content of the infusions disclosed in DE 33 33 719 A1 can carry from 0.1 to 90%. The lactic acid content of the solution to be administered can be from 0.1 to 10%. However, it has emerged in practice that solutions which, besides ciprofloxacin lactate, comprise a free lactic acid concentration of from 0.1 to 90% have only moderate physiological tolerability. Thus, indurations, swellings and reddenings of the injection site occur, the plasma, urea and creatine levels are elevated, and tubular renal lesions result.

EP-A-0 219 784 attempts to avert the problems arising according to DE 33 33 719 A1 (corresponds to EP-A-0 138 018) by providing ciprofloxacin infusions which comprise from 0.015 to 0.5 g of the active ingredient per 100 ml of aqueous solution and, depending on the active ingredient concentration, from 0.9 to 5.0 mol, based on 1 mol of active ingredient, of one or more physiologically tolerated acids. The infusions disclosed in EP-A-0 219 784 comprise, besides the active ingredient, water and other conventional formulation auxiliaries, one or more acids from the group consisting of hydrochloric acid, methanesulfonic acid, propionic acid, succinic acid, glutaric acid, citric acid, fumaric acid, maleic acid, tartaric acid, glutamic acid, gluconic acid, glucuronic acid, galacturonic acid, ascorbic acid, phosphoric acid, adipic acid, hydroxyacetic acid, sulfuric acid, nitric acid, acetic acid, malic acid, L-aspartic acid and lactic acid, in an amount sufficient to dissolve the active ingredient and stabilize the solution. Although it is possible in the manner disclosed in EP-A-0 219 784 to provide infusions of lower toxicity, because if the isoprofloxacin concentration is kept below 0.5% w/v the amount of acid necessary for stabilization can be reduced below the value of 0.1%, which is the minimal amount stated in DE 33 33 719 A1, the infusions disclosed in EP-A-0 219 784 are still just as much in need of improvement in relation to their stability on storage as in relation to reducing the amount of acidic recipients employed.

In this connection, reducing the amount of acid to be employed for stabilization is of fundamental interest, especially when infusions are of comparable stability.

EP-A-0 287 926 relates to solutions of quinolonecarboxylic acids, including ciprofloxacin, which can be administered parenterally, it being proposed to improve the stability on storage by employing particularly pure major active ingredient components. In particular, EP-A-0 287 926 relates to solutions which can be administered parenterally and contain not more than 1 to 10 ppm, based on the major active ingredient component (ciprofloxacin) of the solution, of secondary components (“impurities” of the active ingredient). The reduction of the secondary components introduced by the major active ingredient into the infusion from the outset makes it possible according to EP-A-0 287 926 to reduce the sedimentations from the infusions (turbidities during storage). However, the reduction in the secondary components is a relatively complicated operation.

Even if very pure active ingredients are employed to produce ciprofloxacin infusions, a certain number of particles is found on storage, despite filtration of the solutions after their production—generally through filters with a pore width of 0.2 μm—and after sterilization. According to EP-A-0 287 926, these particles may be produced from a solution in particular by precipitation of the active ingredient or of polycondensation products.

It is further disclosed in the publication DE 197 03 023 A that the number of detectable particles reduce through the use of glass bottles which have a silicone coating on the internal surface. It is possible in this way to improve the storability of high-purity infusions further. It can therefore be assumed that the formation of precipitates is attributable to the number of particles inherently present. The more particles which are present, the more new particles which are formed. This results in an increased rate of particle formation over time.

Both the process described in EP-A-0 287 926 for producing high-purity infusions, and the use of special glass bottles are associated with high expenditure. It must be taken into account in this connection that only a combination of the two variants leads to a worthwhile result.

It would therefore also be advantageous to provide infusions in which it is also possible to employ less pure major active ingredients successfully. The purity of the active ingredients is in this case still to be in the medically acceptable range, although a tolerable amount of secondary substances in this acceptable range is to be as large as possible without leading to infusions which are not stable on storage. Stability on storage means in this connection the absence of sedimentations or precipitates during storage times which are suitable in practice and thus appropriately long.

The aforementioned objects, and other objects which are not explicitly mentioned but which can be inferred directly from the introductory discussion of the prior art or emerge automatically, are achieved by infusions having all the features of the independent product claim. Preferred embodiments of the infusions of the invention are the subject of claims related back to claim 1.

Infusions of 1-cyclopropyl-6-fluoro-1,4-dihydro-4-oxo-7-(1-piperazinyl)quinoline-3-carboxylic acid (ciprofloxacin) obtainable by mixing from 0.015 to 0.5 g of the active ingredient per 100 ml of aqueous solution with sulfuric acid or with sodium hydrogen sulfate in an amount sufficient to dissolve the active ingredient and to stabilize the solution, of 0.96 mol or less than 0.96 mol based on 1 mol of active ingredient, are surprisingly stable on storage, allow the acid content in the ciprofloxacin infusions to be reduced to substoichiometric values and permit the use of ciprofloxacin active ingredients with distinctly higher but still physiologically tolerable amounts of secondary component fractions of more than 10 ppm, without the occurrence of sedimentations from or turbidities of the ciprofloxacin infusions.

It was in this connection to be regarded as particularly surprising that this result was obtainable with sulfuric acid or sodium hydrogen sulfate as acidic excipient. EP-A-0 219 784 lists inter alia lactic acid, phosphoric acid, adipic acid, hydroxyacetic acid and sulfuric acid as possible excipients in an amount of from 0.9 to 5 mol per 1 mol of active ingredient, but these alternatives are completely missing from EP-A-0 219 748 B1, and the amount of acid(s) is, according to EP-0-219 784 B, to be between 1.04 and 2.2,mol per 1 mol. The results obtained with hydroxyacetic acid and phosphoric acid in sub stoichiometric amounts are useless. To this extent, successful use of sulfuric acid was not obvious, at least not in the amount of 0.96 mol or less per 1 mol of active ingredient which is essential to the invention.

Infusion solutions of particular interest according to the present invention are those wherein the total content of sulfuric acid is 0.9 mol or less based on 1 mol of ciprofloxacin (active ingredient). Infusions of total content of sulfuric acid of 0.8 mol or less are even expedient. Very particular preference is furthermore given to infusions in which the total content of sulfuric acid is 0.6 mol or less than 0.6 mol per 1 mol of active ingredient.

A derivative of sulfuric acid has also proved suitable as acidic excipient. This is sodium hydrogen sulfate. The latter can be employed for example as aqueous solution for dissolving ciprofloxacin, it being possible to prepare the aqueous solution of sodium hydrogen sulfate in situ.

In an expedient variant, infusions of the invention can be obtained by admixing the active ingredient with sulfuric acid in an amount sufficient to dissolve the active ingredient and to stabilize the solution, of 0.96 mol or less per 1 mol of active ingredient, and an amount of NaOH which is equimolar to the amount of sulfuric acid. The amount of sulfuric acid in this case is preferably in the range from 0.96 mol to 0.93 mol per 1 mol of the active ingredient. These ratios of amounts result in complete and clear solutions which are exceptionally stable on storage. Below 0.93 mol of sodium hydrogen sulfate per mol of active ingredient the storage stability may be slightly impaired.

It is also possible as an alternative to preparation of the solution of sodium hydrogen sulfate in situ, it is also possible in another expedient variant to start directly from sodium hydrogen sulfate. In this case it is preferred in turn for the amount of sodium hydrogen sulfate to be in the range from 0.96 mol to 0.93 mol per 1 mol of the active ingredient. An optimum emerges when the amount of sodium hydrogen sulfate in the solution is less than 0.95 mol per 1 mol of the active ingredient.

It has also emerged, very unexpectedly, that the use of sulfuric acid or sodium hydrogen sulfate together with certain additional acids enables ciprofloxacin infusions in which the stabilization is superadditive, almost synergistic. Particular infusions are in this regard characterized in that they comprises as additional acid, besides sulfuric acid, a mono- or diester of orthophosphoric acid with glycerol or a higher-grade physiologically tolerated sugar such as glucose, sucrose, fructose or sugar alcohols such as sorbitol, mannitol or xylitol, where the total content of sulfuric acid and additional acid is less than 1.04 mol per 1 mol of active ingredient. This makes it possible to provide infusions which are even more stable on storage and which are additionally less prone to sedimentations or turbidities, even if the content of secondary components from the active ingredient is smaller than 50 ppm.

It is particularly preferred for the acid to be employed for the additional stabilization of the ciprofloxacin infusions to comprise glycerol esters of orthophosphoric acid. Monoglycerol orthophosphoric esters are particularly preferred. Maximum interest attaches to infusions in which, besides sulfuric acid, the acid(s) is or are glycerol 1-phosphate, glycerol 2-phosphate or a mixture of the glycerol phosphoric monoesters. In addition, diphosphates are also particularly suitable acids. These include, in particular, glucose diphosphate and fructose 1,6-diphosphate. Said acids are approximately as strong as phosphoric acid. However, additions of glycerol 1-phosphate, glycerol 2-phosphate, glucose diphosphate and/or fructose 1,6-diphosphate are clearly superior to phosphoric acid in relation to the improvement in the stability on storage.

The minimum amount of acid necessary per mol of active ingredient for dissolving depends, of course, also on the active ingredient concentration and the acid(s) used and is thus not constant. Account must also be taken of the fact that the statements in the amounts of acids relate only to the amounts which, according to the generally known basic laws of chemistry, are not converted into the corresponding salt(s) through addition of bases. Dissociation of the acids has been ignored in the statements of amounts, so that they relate to the dissociated and undissociated amount of acid.

The infusions of the invention may also comprise other formulating agents such as complexing agents, antioxidants, tonicity agents and/or agents to adjust the pH. The osmolality of the infusions is from 0.20 to 0.70 osm/kg, preferably 0.26 to 0.39 osm/kg, and is adjusted by tonicity agents such as NaCl, mannitol, glucose, sucrose and glycerol or mixtures of such substances. It is also possible where appropriate to employ for this purpose substances which are present in conventional, commercially available infusion vehicle solutions.

Customary infusion vehicle solutions include infusions with addition of electrolytes without carbohydrates, such as sodium chloride solution, Ringer's lactate solution and others, and those with carbohydrates, and solutions to supply amino acids, in each case with or without a carbohydrate content. Examples of such infusion vehicle solutions are listed in the Rote Liste 1998, Verzeichnis von Fertigarzneimitteln der Mitglieder des Bundesverbandes der Pharmazeutischen Industrie e.V., Editio Cantor, Aulendorf/Württ.

Preferred infusions comprise, besides water, active ingredient and other formulation aids, an amount of sodium chloride or other conventional tonicity excipients such as to provide a solution which is isotonic with the tissue fluid of the human or animal body or is slightly hypo- or hypertonic.

The infusions of the invention have a pH of from 2.6 to 5.2, preferably 3.0 to 5.2. pH values of from 3.6 to 4.7 or 3.9 to 4.5 are likewise preferred. Very particularly preferred pH values are in the range from 4.1 to 4.3.

The infusions of the invention can be in the form of dosage units suitable for infusion, with extractable contents of from 40 to 600 ml, preferably 50 to 120 ml.

The present invention additionally relates to a process for producing infusions as claimed in the independent claim relating to the infusions, which comprises adding an amount of sulfuric acid or an amount of sulfuric acid and NaOH or an amount of sodium hydrogen sulfate, and in each case where appropriate an amount of a physiologically tolerated monoester or diester of orthophosphoric acid or of a mixture of a plurality of physiologically tolerated monoester or diester derivatives of orthophosphoric acid, to a suitable amount of the active ingredient, where appropriate in the form of a salt such as alkali metal or alkaline earth metal salt or addition salt, of a hydrate or of a hydrate of the salt or in the form of mixtures of these salts or hydrates, where the amount of acid totals 0.96 mol or less than 0.96 mol per 1 mol of active ingredient, adding formulation aids where appropriate, and making up with water or a conventional infusion vehicle solution in such a way that a concentration range of from 0.015 to 0.5 g is set up for the active ingredient, where on use of an alkali metal or alkaline earth metal salt of the active ingredient the amounts of acid necessary for dissolving additionally contain the amounts necessary to neutralize the active ingredient anion, and on use of an addition salt part of the amounts of acid necessary is already present in the active ingredient to be employed.

Care must be also be taken in the production that the solutions comply with the properties already mentioned in relation to pH, amounts of acid and osmolalities. In the case where the active ingredient is used in salt form it is expediently possible to employ an acid whose anion corresponds to the anion of the active ingredient salt or salt hydrate.

The pH of the infusions of the invention can be adjusted with (physiologically) tolerated acids and/or bases to the abovementioned values, namely 2.6 to 5.2, expediently 3.0 to 5.2, and particularly 3.6 to 4.7. To expedite the production process, especially the dissolving of solid components, it is possible for the solutions or only a part thereof to be heated slightly, preferably to temperatures between 20° C. and 80° C.

The solutions of the invention can be produced particularly economically via concentrated solutions. For this purpose, the amount of active ingredient required for a batch is dissolved with the major amount of acid necessary for the complete batch (for example 95% based on moles) in a little water—where appropriate with heating. This concentrate is then subsequently diluted. After dilution, any other excipients—such as, for example, sodium chloride as tonicity agent—are added as is the amount of acid still lacking where appropriate.

The solution is generally filtered after production thereof in order to remove most of the particles.

Suitable filtration methods are known per se, so that reference may be made to the prior art. The number of particles is in this case limited to the medically necessary and economically worthwhile. These data and suitable methods are disclosed in text books.

After filtration of the solution, it can be dispensed into suitable containers. Without intending to effect a restriction hereby, in general glass bottles or bags made of plastic sheets which are suitable for medical use are employed for this purpose. Polyolefin-based PVC-free bags are particularly preferred. To improve storability, these bags can, where appropriate, be provided with a further outer packaging.

The solutions of the invention show high stability on storage, which is not limited by the number of particles. It is possible to dispense with the effort described in the documents EP 0 287 926 and DE-A-197 30 23 in order to make the solutions stable.

### EXAMPLES

The following examples and comparative examples serve to explain the invention in more detail without intending thereby to effect a restriction. Water for infusions was employed to prepare the solutions.

### Example 1

3 mmol (1 g) of ciprofloxacin were suspended in 250 ml of water. 196.5 ml of a sulfuric acid solution which was obtained by diluting 15.5 ml of sulfuric acid (c=0.1 mol/l; Merck AG) with 181 ml of water were added to this suspension. The total amount of sulfuric acid added was 1.5 mmol. The addition took place over a period of 2 hours, during which the pH did not fall below 3.0. A clear solution with a pH of 4.5 was obtained. This solution was then mixed with 50 ml of an NaCl solution which contained 4.41 g of NaCl, and then diluted with water to 500 ml with water.

The solution obtained in this way was filtered, dispensed into a glass bottle for medical purposes and then sterilized at 121° C. The sterile solution obtained in this way was stored at room temperature and regularly checked visually for 6 months. After this period, no changes were found visually.

The subvisual particles, determined by the usual method of the light block procedure, were low and likewise remained unchanged. They complied with the specifications laid down in the Ph. Eur. for such solutions.

### Example 2

Example 1 was essentially repeated. However, the resulting solution was dispensed not into a glass bottle but into a polyolefin-based bag which is likewise suitable for medical purposes.

After storage over a period of 6 months, no changes were found visually.

The subvisual particles, determined by the usual method of the light block procedure, were low and likewise remained unchanged. They complied with the specifications laid down in the Ph. Eur. for such solutions.

### Comparative Example 1

3 mmol (1 g) of ciprofloxacin were suspended in 250 ml of water. A solution of 2.9 mmol (0.22 g) of hydroxyacetic acid in 200 ml of water was slowly added to this suspension. The suspension did not dissolve completely. Checking of the storability was thus obsolete.

Comparative Example 2

3 mmol (1 g) of ciprofloxacin were suspended in 250 ml of water. A solution of 2.9 mmol (0.26 g) of lactic acid in 200 ml of water was slowly added to this suspension. The suspension did not dissolve completely. Checking of the storability was thus obsolete.

### Comparative Example 3

3 mmol (1 g) of ciprofloxacin were suspended in 250 ml of water. A solution of 3 mmol of phosphoric acid in 200 ml of water was slowly added to this suspension. The addition took place over a period of 2 hours, during which the pH did not fall below 3.0. A clear solution was obtained. This solution was then mixed with 50 ml of an NaCl solution which contained 4.41 g of NaCl, and then diluted with water to 500 ml.

The solution obtained in this way was filtered as in example 1, dispensed into a glass bottle for medical purposes and then sterilized at 121° C. The sterile solution obtained in this way was stored at room temperature and regularly checked visually for 2 months. A visually detectable formation of crystals occurred after 2 months. The experiment was then discontinued.

### Example 3

5.7 mmol of 100% pure sulfuric acid (analytical grade) were slowly mixed with 5.7 mmol of NaOH (analytical grade) in 500 ml of double-distilled water. 6 mmol of ciprofloxacin base were suspended in 500 ml of water. This suspension was slowly added to a sodium hydrogen sulfate solution. The addition took place over a period of 1 h, the pH of the resulting solution initially being about 2.7. After equilibration at about 40° C. for 60 minutes and complete dissolution of the active ingredient, the solution had a pH of 4.9. A clear solution was obtained.

The solution obtained in this way was filtered as in example 1, dispensed into a glass bottle for medical purposes and then sterilized at 121° C. The sterile solution obtained in this way was stored at room temperature and regularly checked visually for 2 months. No visually detectable formation of crystals occurred. The glass bottles were visually satisfactory.

### Example 4

As example 3 with the difference that the resulting solution was stored in a plastic bag as in example 2. The visual assessment of the bag gave rise to no criticisms at all.

The tests and comparative tests clearly show that solutions with a substoichiometric acid/ciprofloxacin ratio are obtainable only on use of sulfuric acid. The solutions of the invention can be stored for a long period without problems arising in relation to the stability. In contrast to this, prior art infusions form precipitates. This is shown quite clearly in particular by comparative test 3. This problem has previously been solved by using high-purity ciprofloxacin solutions as described in the documents EP-0 287 926 and DE-A-197 30 23. Such measures can be dispensed with on use of sulfuric acid.

It is true that, from the medical viewpoint, solutions having a minimal particle content are always to be preferred. However, for economic reasons, the purification must stay within limits. It should be taken into account in this connection that particles may possibly likewise get into the solution on administration of the infusion through tubing etc. There is thus no reason to reduce the particle content below a certain number borne even by economic considerations, as long as they comply with the limits for the visual and subvisual particles as laid down in appropriate pharmacopeias.

It therefore remains to be stated that the particles which might in some circumstances be responsible for limited stability of the ciprofloxacin solutions obtained using lactic acid or phosphoric acid do not lead to formation of precipitate or subvisual particles on use of sulfuric acid.

It remains to be stated that the solutions of the invention remain stable. Sedimentations like those described for the lactic acid solutions in EP-A-0 287 926 as polycondensation products are obviously not formed. Efforts like those described in EP 0 287 926 or DE-A-197 30 23 to obtain stable solutions are accordingly unnecessary. This unexpected result makes an inexpensive method possible for producing ciprofloxacin solutions which are stable in the long term.

1. Infusions of 1-cyclopropyl-6-fluoro-1,4-dihydro-4-oxo-7-(1-piperazinyl)quinoline-3-carboxylic acid (ciprofloxacin) which are stable on storage and are obtainable by mixing from 0.015 to 0.5 g of the active ingredient per 100 ml of aqueous solution and sulfuric acid or sodium hydrogen sulfate in an amount sufficient to dissolve the active ingredient and to stabilize the solution, of 0.96 mol or less per 1 mol of active ingredient.

2. Infusions as claimed in claim 1, characterized in that the total content of sulfuric acid is 0.9 mol or less based on 1 mol of active ingredient.

3. Infusions as claimed in claim 1, characterized in that the total content of sulfuric acid is 0.8 mol or less based on 1 mol of active ingredient.

4. Infusions as claimed in claim 1, characterized in that the total content of sulfuric acid is 0.6 mol or less based on 1 mol of active ingredient.

5. Infusions as claimed in claim 1, obtainable by admixing the active ingredient with sulfuric acid in an amount sufficient to dissolve the active ingredient and to stabilize this solution, 0.96 mol or less per 1 mol of active ingredient, and with an amount of NaOH equimolar to the amount of sulfuric acid.

6. Infusions as claimed in claim 5, characterized in that the amount of sulfuric acid is in the range from 0.96 mol to 0.93 mol per 1 mol of the active ingredient.

7. Infusions as claimed in claim 1, characterized in that the amount of sodium hydrogen sulfate is in the range from 0.96 mol to 0.93 mol per 1 mol of the active ingredient.

8. Infusions as claimed in claim 1, characterized in that the amount of sodium hydrogen sulfate is less than 0.95 mol per 1 mol of the active ingredient.

9. Infusions as claimed in any of the preceding claims, characterized in that they comprises as additional acid a mono- or diester of orthophosphoric acid with glycerol or a higher-grade physiologically tolerated sugar such as glucose, sucrose, fructose or sugar alcohols such as sorbitol, mannitol or xylitol, where the total content of sulfuric acid or sodium hydrogen sulfate and additional acid is less than 1.04 mol per 1 mol of active ingredient.

10. Infusions as claimed in claim 9, characterized in that the additional acid is glycerol 1-phosphate, glycerol 2-phosphate, glucose diphosphate and/or fructose 1,6-diphosphate.

11. Infusions as claimed in one or more of the preceding claims, characterized in that they comprise complexing agents, antioxidants, tonicity agents and/or agents for adjusting the pH as formulation aids.

12. Infusions as claimed in one or more of the preceding claims, characterized in that they have an osmolality of from 0.20 to 0.70 osm/kg and comprise tonicity agents such as NaCl, sorbitol, mannitol, glucose, sucrose, xylitol, fructose, glycerol or mixtures of such substances and/or, where appropriate, substances which are present as ingredients in conventional infusion vehicle solutions.

13. Infusions as claimed in claims 1 to 12, characterized in that they have a pH in the range from 2.6 to 5.2, expediently 3.0 to 5.2, preferably in the range from 3.6 to 4.7, more preferably in the range from 3.9 to 4.5, particularly preferably in the range from 4.1 to 4.3.

14. Infusions as claimed in any of the preceding claims, characterized in that, besides active ingredient, water and other formulation aids, they comprise an amount of sodium chloride or other excipients customary as tonicity agents such as to provide a solution which is isotonic with the tissue fluid of the human or animal body or is slightly hypo- or hypertonic.

15. Infusions as claimed in one or more of the preceding claims, characterized in that, besides active ingredient, water and other formulation aids, they comprise 0.96 mol of a mixture of glycerol 1-phosphate and glycerol 2-phosphate (based on 1 mol of active ingredient) and from 0.6 to 2.2 mol of NaCl based on 100 ml of solution.

16. Infusions as claimed in claims 1 to 15 in dosage units which are suitable for infusion and which can be produced either from glass or from plastics suitable for this purpose, with extractable contents of from 40 to 600 ml, preferably from 50 to 120 ml.

17. A process for producing infusions as claimed in claims 1 to 16, characterized in that an amount of sulfuric acid or an amount of sulfuric acid and NaOH or an amount of sodium hydrogen sulfate, and in each case where appropriate an amount of a physiologically tolerated monoester or diester of orthophosphoric acid or of a mixture of a plurality of physiologically tolerated monoester or diester derivatives of orthophosphoric acid, is added to a suitable amount of the active ingredient, where appropriate in the form of a salt such as alkali metal or alkaline earth metal salt or addition salt, of a hydrate or of a hydrate of the salt or in the form of mixtures of these salts or hydrates, where the amount of acid totals 0.96 mol or less per 1 mol of active ingredient, formulation aids are added where appropriate, and making up with water or a conventional infusion vehicle solution is done in such a way that a concentration range of from 0.015 to 0.5 g is set up for the active ingredient, where on use of an alkali metal or alkaline earth metal salt of the active ingredient the amounts of acid necessary for dissolving additionally contain the amounts necessary to neutralize the active ingredient anion, and on use of addition salts part of the amounts of acid necessary is already present in the active ingredient salt to be employed.

18. The process as claimed in claim 17, characterized in that the pH of the fusion solution is adjusted to 3.0 to 5.2 with (physiologically) tolerated buffer systems.

19. The process as claimed in claim 17 or 18, characterized in that the infusions are produced by heating.

20. The use of infusions and/or other dosage forms which are converted before administration into the infusions as claimed in claims 1 to 16 for producing dosage units suitable for infusion with extractable contents of from 40 to 600 ml.

21. Aqueous infusion solution of 1-cyclopropyl-6-fluoro-1,4-dihydro-4-oxo-7-(1-piperazinyl)quinoline-3-carboxylic acid (ciprofloxacin) which is stable on storage and is obtained by mixing (a) from 0.015 g to 0.5 g of the ciprofloxacin per 100 ml of aqueous solution and (b) sodium hydrogen sulfate in an amount sufficient to dissolve the ciprofloxacin and to stabilize the solution equal to 0.96 mol or less per mol of active ingredient.

22. Infusion solution of claim 21, obtained by admixing the ciprofloxacin with sulfuric acid in an amount sufficient to dissolve the active ingredient and to stabilize this solution equal to 0.96 mol or less per mol of active ingredient, and with an amount of NaOH equimolar to the amount of sulfuric acid.

23. Infusion solution of claim 22, wherein the amount of sulfuric acid is in the range from 0.93 mol to 0.96 mol per mol of the ciprofloxacin.

24. Infusion solution of claim 21, wherein the amount of sodium hydrogen sulfate is in the range from 0.93 mol to 0.96 mol per mol of the ciprofloxacin.

25. Infusion solution of claim 21, wherein the amount of sodium hydrogen sulfate is less than 0.95 mol per mol of the ciprofloxacin.

26. Infusion solution of claim 21, comprising as additional acid, a mono- or diester of orthophosphoric acid with glycerol or a higher-grade physiologically tolerated sugar or sugar alcohols, where the total content of sodium hydrogen sulfate and additional acid is less than 1.04 mol per mol of ciprofloxacin.

27. Infusion solution of claim 26, wherein the additional acid is selected from the group consisting of glycerol 1-phosphate, glycerol 2-phosphate, glucose diphosphate and fructose 1,6-diphosphate.

28. Infusion solution of claim 26, comprising at least one of glucose, sucrose, and fructose.

29. Infusion solution of claim 26, comprising at least one of sorbitol, mannitol, and xylitol.

30. Infusion solution of claim 21, comprising at least one member selected from the group consisting of complexing agents, antioxidants, tonicity agents and agents for adjusting the pH as formulation aids.

31. Infusion solution of claim 30, having an osmolality of from 0.20 to 0.70 osm/kg and comprising at least one tonicity agent.

32. Infusion solution of claim 31, wherein said tonicity agent is selected from the group consisting of NaCl, sorbitol, mannitol, glucose, sucrose, xylitol, fructose, glycerol or mixtures thereof.

33. Infusion solution of claim 21, having a pH in the range from 2.6 to 5.2.

34. Infusion solution of claim 21 having a pH in the range from 3.0 to 5.2.

35. Infusion solution of claim 21 having a pH in the range from 3.6 to 4.7.

36. Infusion solution of claim 21 having a pH in the range from 3.9 to 4.5.

37. Infusion solution of claim 21 having a pH in the range from 4.1 to 4.3.

38. Infusion solution of claim 21, comprising an amount of an excipient effective as a tonicity agent to provide a solution which is substantially isotonic with the tissue fluid of a human body or animal body.

39. Infusion solution of claim 38, wherein the solution is slightly hypotonic.

40. Infusion solution of claim 38, wherein the solution is slightly hypertonic.

41. Infusion solution of claim 38, wherein said tonicity agent is sodium chloride.

42. Infusion solution of claim 21 in glass or plastic dosage units which are suitable for infusion with extractable contents of from 40 ml to 600 ml.

43. Infusion solution of claim 42, comprising extractable contents of 50 ml to 120 ml.

44. A process for producing an aqueous infusion solution of claim 21, comprising adding an amount of sulfuric acid and NaOH or an amount of sodium hydrogen sulfate to a suitable amount of ciprofloxacin, where the amount of acid totals 0.96 mol or less per mol of ciprofloxacin, formulation aids are added where appropriate, and diluting to a concentration range of from 0.015 g to 0.5 g of ciprofloxacin per 100 ml of solution.

45. The process of claim 44, comprising adjusting the pH of the infusion solution to 3.0 to 5.2 with (physiologically) tolerated buffer systems.

46. The process of claim 44, comprising producing the infusion solution by heating.

47. The process of claim 44, comprising adding an amount of physiologically tolerated monoester or diester of orthophosphoric acid or of a mixture of a plurality of physiologically tolerated monoester or diester derivatives of orthophosphoric acid.

48. The process of claim 44, wherein the ciprofloxacin is in the form of a salt, a hydrate, a hydrate of a salt, or a mixture thereof.

49. The process of claim 44, wherein the ciprofloxacin is in the form of an alkali metal salt, an alkaline earth metal salt, or an addition salt.

50. The process of claim 49, wherein the ciprofloxacin is in the form of an alkali metal salt or an alkaline earth metal salt, and sufficient acid is added to dissolve the ciprofloxacin and to neutralize anions of the ciprofloxacin.

51. The process of claim 49, wherein the ciprofloxacin is in the form of an addition salt comprising a portion of the acid to be added.

52. The process of claim 44, comprising adding a formulation agent.
